(19) United States
(12) Patent Application Publication
(10) Pub. No.: US 2026/0115727 A1
(43) Pub. Date: Apr. 30, 2026

(54) URINE ANALYSIS CARTRIDGE

(71) Applicant: Withings, ISSY LES MOULINEAUX (FR)

(72) Inventors: Victor GEFFRAYE, ISSY LES MOULINEAUX (FR); Pierre-Arnaud DONNET, ISSY LES MOULINEAUX (FR)

(21) Appl. No.: 19/341,812

(22) Filed: Sep. 26, 2025

(30) Foreign Application Priority Data
Sep. 27, 2024 (FR) FR2410350

(51) Int. Cl. B01L 3/00; G01N 33/493

(52) U.S. Cl. CPC B01L 3/5085; G01N 33/493; B01L 2300/0609; B01L 2300/087; B01L 2300/105

(57) ABSTRACT

A urine analysis cartridge includes a support and a plurality of moisture-tight chambers, each chamber being adapted to receive urine for analysis. The plurality of moisture-tight chambers is held in position by the support. At least a portion of the support, called the desiccant portion, is composed of a solid desiccant, capable of absorbing moisture from the air in contact with the desiccant portion, the desiccant portion being outside the moisture-tight chambers.

### CROSS-REFERENCE TO RELATED APPLICATIONS

This application claims priority to French Patent Application No. FR2410350, filed Sep. 27, 2024, the entire content of which is incorporated herein by reference in its entirety.

### FIELD

The present invention relates to a cartridge for urine analysis. The invention also relates to a urine analysis device comprising such a cartridge and a station configured to be positioned on a wall of a toilet bowl.

### BACKGROUND

Many biological parameters are reflected in an individual's urine. For example, a urine sample can be used to detect health problems such as urinary tract infection, diabetes or kidney failure. A urine sample can also reflect the quality of a diet, identify a fertile period or pregnancy, and detect the use of drugs or tobacco. In these cases, it makes sense to monitor various biological parameters periodically.

Document WO2021/175909 describes a device that fits onto the wall of a toilet bowl and collects a urine sample prior to optical analysis. The device comprises a station and a cartridge that can be removed and replaced from the station. The cartridge contains urine-reactive reagents, including urine test strips. These reagents are arranged in respective chambers. Document EP4338839 describes a cartridge with particularly tight chambers to preserve the life of the reagents, which are sensitive to moisture.

In order to extend the life of moisture-sensitive reagents, it is known to place desiccant in contact with these reagents. For example, documents U.S. Pat. Nos. 6,497,845B1 and 5,575,403A describe desiccants arranged in reagent-accommodating housings.

### SUMMARY

The purpose of the present description is to further improve the operation and service life of the urine analysis device by limiting moisture in the device.

To this end, an aspect of the present description concerns a cartridge for urine analysis comprising: a support; a plurality of moisture-tight chambers, each moisture-tight chamber being suitable for receiving urine for urine analysis; wherein: the plurality of moisture-tight chambers is held in position by the support; at least a portion of the support, called the desiccant portion, is composed of a solid desiccant, capable of absorbing moisture from the air in contact with the desiccant portion, the desiccant portion being outside the moisture-tight chambers.

In fact, the cartridge may comprise several dozen test strips. To perform a urine analysis on the user, the station injects urine into a chamber. An analysis device can then analyze the result of this reaction and inform the user. For each analysis, urine is injected into one of the chambers, which is then pierced. The inventors realized that these repeated injections increase moisture in the station, which can lead to a harmful environment for the proper operation of the electronic devices present in the station and the life of the urine analysis device.

To this end, the cartridge support according to the present description offers a dual function enabling it both to mechanically support the chambers to hold them in position and also to absorb some of the moisture present in the station when the cartridge is in place in the station. Through this dual function, the structural parts serve as desiccant, thereby maximizing absorption volume. In fact, there is a strong space constraint in the station that does not allow the addition of an extra desiccant volume. In addition, the desiccant portion can be positioned so that it does not interfere with urine analysis. Lastly, as the cartridge is a consumable and replaceable element, replacing the cartridge also enables the desiccant portion to be replaced, thus renewing the desiccant's absorption capacity.

By moisture-tight chamber, it is meant at least moisture-tight chamber.

By “outside the moisture-tight chambers”, it is meant that the desiccant portion is not in contact with the inside of the moisture-tight chambers.

In an embodiment, each moisture-tight chamber houses a reagent configured to react with urine.

In an embodiment, the solid desiccant is a hygroscopic material.

In an embodiment, the solid desiccant is a bi-material mixture of a plastic polymer and a desiccant material.

In an embodiment, the solid desiccant is configured to absorb water vapor molecules upon contact with the substrate.

In an embodiment, the solid desiccant is formed from zeolite-filled polypropylene.

In an embodiment, the desiccant portion has an absorption capacity between 100 mg/g and 300 mg/g, particularly between 140 mg/g and 200 mg/g.

In an embodiment, the desiccant portion represents at least 25%, for example 50%, of the surface support.

In an embodiment, the desiccant portion is rigid.

In an embodiment, the desiccant portion comprises a zone for receiving the force of a station actuator.

In an embodiment, the desiccant portion forms a base on which the chambers are mounted.

In an embodiment, each chamber comprises a pierceable cover, for example by a needle.

In an embodiment, the force-receiving zone is a hub and the actuator comprises a rotating shaft.

In an embodiment, the cover is transparent or translucent.

In an embodiment, each reagent is arranged on a test strip.

In an embodiment, the support is a rotating support.

In an embodiment, the chambers are arranged side by side in the shape of a right circular cylinder of at least 80% of a circle.

In an embodiment, the cylinder has a diameter between 3 and 10 cm.

In an embodiment, the chambers are arranged on an outer wall of the rotating support.

In an embodiment, each chamber extends in a main direction orthogonal to the base.

In an embodiment, the desiccant portion substantially forms a disk.

In an embodiment, the disk has a diameter between 3 cm and 10 cm.

In an embodiment, the disk has a thickness between 1 mm and 10 mm.

In an embodiment, the cartridge further comprises an additional desiccant, different from the desiccant portion, disposed in at least one chamber.

In an embodiment, the additional desiccant is a strip of desiccant material.

The present description also relates to an optical urine analysis device comprising: a cartridge as defined above; a station configured to be positioned on a wall of a toilet bowl, the station comprising: a housing comprising a receiving compartment in which the cartridge is at least partially received, an injector configured to inject fluid into one of the chambers, and an analysis device for analyzing urine injected into the chamber.

In an embodiment, the cartridge is removably arranged in the housing.

In an embodiment, the analysis device comprises a light source and a light sensor, configured to emit and receive light.

In an embodiment, the analysis device is configured to analyze a color change of the reagent upon contact with the injected urine.

In an embodiment, the injector is arranged in a zone which is, when the cartridge is mounted in the station, radially internal to the cartridge.

In an embodiment, there is provided a urine-analysis cartridge comprising: a support structure; and a plurality of moisture-tight chambers carried by the support structure, each chamber enclosing an interior volume adapted to receive urine for analysis and being initially sealed by a pierceable cover; wherein the support structure includes a desiccant portion formed of a solid desiccant material configured to sorb water vapor from ambient air in contact therewith; and wherein the desiccant portion is external to the interior volumes of the moisture-tight chambers and is not in fluid communication with any of the interior volumes while the pierceable covers remain intact, whereby the support structure both mechanically holds the moisture-tight chambers in position and reduces ambient humidity in an associated analysis station when the cartridge is installed.

In an embodiment, the desiccant portion defines a force-receiving zone comprising a hub configured to couple with a drive member of an analysis station to transmit rotational and/or translational motion that indexes the chambers past an injector and/or an analysis unit, the hub being formed of the solid desiccant material such that the force-receiving zone simultaneously provides mechanical coupling and ambient-humidity sorption.

### BRIEF DESCRIPTION OF THE DRAWINGS

Further features, details and benefits will become apparent from the detailed description below, and from analysis of the appended drawings, in which:

FIG. 1 shows a simplified schematic representation of a urine analysis device installed in a toilet bowl,

FIG. 2 shows an exploded view of the urine analysis device, in which the station and cartridge are visible,

FIG. 3 shows a detailed view of a cartridge according to one embodiment in cooperation with an actuator of the station,

FIG. 4 shows an exploded perspective view of the bottom of the cartridge shown in FIG. 3,

FIG. 5 shows an exploded top perspective view of the cartridge of FIG. 3,

FIG. 6 shows a cross-sectional view of a cartridge and a station in an embodiment, at the location of an optical analysis device in the station,

FIG. 7 shows a perspective view of a cartridge according to another embodiment in cooperation with an actuator of the station, and

FIG. 8 shows a perspective view of a cartridge according to another embodiment in cooperation with an actuator of the station.

### DETAILED DESCRIPTION

The present description presents various examples of a urine analysis device comprising a station as disclosed in documents WO2021/175909 and WO2021/175944, hereinafter referred to as WO′909 and WO′944. Variations of the stations are presented in documents WO2023036805, WO2023036806, WO2023036808, WO2023036809, hereinafter referred to as WO′80X.

The following paragraphs explain the general principle of a urine analysis device, but all the details of documents WO′909 and WO′933 (as well as all the above-mentioned PCT documents) are applicable.

### General Shape of the Housing

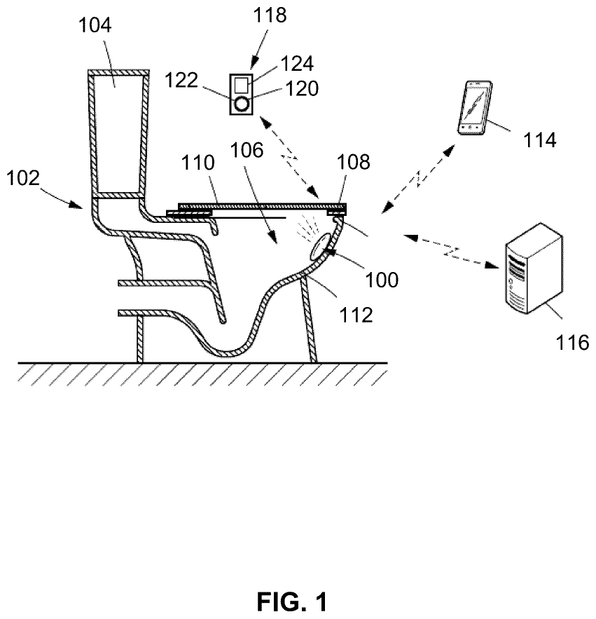

FIG. 1 schematically illustrates an analysis device 100 (hereinafter also referred to as “device 100”) for urine analysis installed in toilet 102. Toilets 102 typically comprise a water tank 104, a bowl 106, a seat 108 and a seat cover 110. The analysis device 100 is configured to be placed entirely within the toilet bowl. By “in the bowl” is meant “placed in the interior volume defined by the bowl”. The analysis device 100 is removably arranged in the toilet 102. For example, the analysis device 100 can be easily removed from the toilet to replace a cartridge, then replaced in the toilet 102. The analysis device 100 is placed on an inner wall 112 of the toilet bowl 106. The analysis device 100 is positioned so that it is generally under a user's urine stream, so that when a user urinates (generally in a seated position), the urine comes into contact with the analysis device 100. The analysis device 100 can communicate remotely with a remote entity, such as smartphone 114 or server 116.

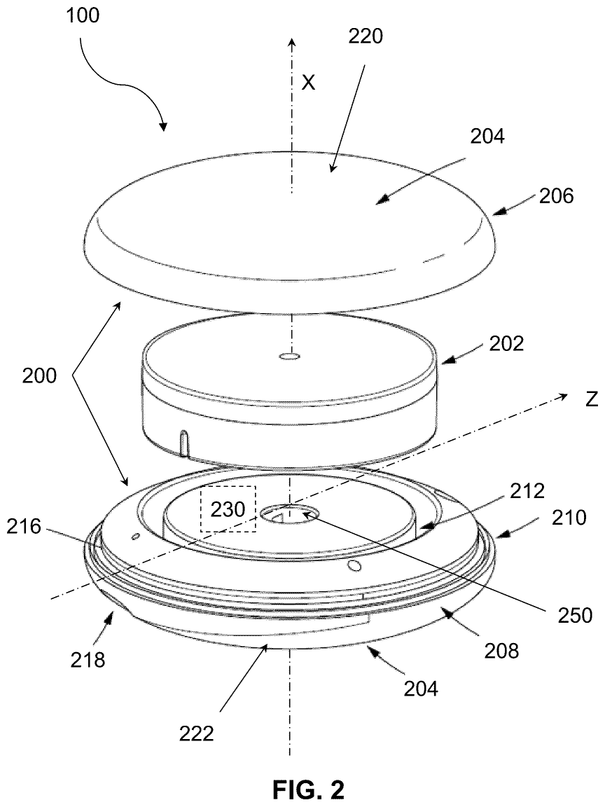

As further illustrated in FIG. 2, the analysis device 100 may comprise a station 200 and a cartridge 202. The cartridge 202 may be removably mounted on the station 200. The station 200 may comprise a housing 204 which may comprise two shells, in particular a front shell 206 and a rear shell 208. The front shell 206 and the rear shell 208 may cooperate with each other via a fastening mechanism 216, in a plane normal to the X axis. The front shell 206 and rear shell 208 can be reversibly assembled, for example by screwing or clipping. Alternatively, the front shell 206 and rear shell 208 can be permanently joined, for example by gluing, clipping, magnetizing or ultrasonic welding. Of course, other fastening means can be used to join the two shells.

In particular, as shown in FIG. 2, the front shell 206 and the rear shell 208 are screwed together. An internal part of the front shell 206 comprises a thread. The thread on the front shell 206 is designed to cooperate with a complementary thread on the inside of the rear shell 208. This allows easy disassembly of the housing 204 to access the test assembly inside the housing.

A seal may be present between the front shell 206 and the rear shell 208. In this way, housing 204 is tight from the outside of device 100.

As can be seen from the figures, housing 204 may have the overall external shape of a circular roller. In other words, housing 204 has a spheroidal shape. The X axis is the center line of the housing. Beneficially, the front shell 206 can be substantially rotationally symmetrical, giving the device an aerodynamic appearance once installed. Housing 204 serves as a urine collector.

The housing 204 comprises a front face 220 for receiving a stream of urine directly from a user urinating on the toilet, and a rear face 222 opposite the front face 220. As illustrated in FIG. 2, the front face 220 can be arranged on the front shell 206 and the rear face 222 can be arranged on the rear shell 208. The front face 220 faces the inside of the bowl 106. The front face 220 is therefore intended to receive urine when the user urinates while sitting on the toilet 102. The rear face 222 faces the inner wall 112 of the bowl 106.

The outer surface of the front face 220 may be smooth. In other words, the front face 220 is free of ridges or grooves. In this way, the flow of urine coming into contact with the front face 220 catches and spreads over the front face 220. The front face 220 may be substantially rotationally symmetrical about the X axis.

The outer surface of housing 204 may also be white or light-colored. The color of the outer surface can be similar to that of the toilet, which enhances the discreetness of the device.

In an embodiment, the housing 204 may have a diameter, measured in the direction orthogonal to the X axis, between 50 mm and 150 mm. In an embodiment, the housing 204 can have a thickness, measured in the direction of the X axis, between 15 mm and 50 mm. In this way, housing 204 is sufficiently compact to be fully housed in the toilet bowl. The urine analysis device 100 is unobtrusive. In addition, housing 204 is large enough to systematically come into contact with the urine received in the toilet bowl. The user can then urinate in the toilet without worrying about the urine analysis device, or alternatively aim approximately.

According to another aspect, in an embodiment, the housing 204 has a general form factor such that the ratio between thickness and diameter is between 0.2 and 0.5, and for example between 0.3 and 0.4. Such proportions are reminiscent of a natural pebble and give the device a soothing appearance. The spheroidal ‘pebble’ shape minimizes splash-back and offers low resistance to water flow, encouraging complete and uniform flushing.

Housing 204 may be made of a hydrophilic material. For example, the material of housing 204 may be a ceramic, a polyamide (PA), a silicone or a hydrophilic polymer. The outer surface of housing 204 can also be treated with a hydrophilic surface treatment, for example Aculon's AcuWet®, a hydrophilic polymer, or Arkema's Pebax®.

### Test Assembly

Station 200 includes a collection port 218, located, for example, on rear shell 208. Collection port 218 is configured to collect urine flowing onto the surface of housing 204. Station 200 may also include a drain port configured to drain liquid out of device 100. The rear-facing collection port and spacer arrangement prevents direct exposure to user urine streams and flush surges, reducing fouling risk and ensuring sensor longevity. This also avoids turbulent flow disruption during sample intake, improving test accuracy.

A test assembly 230 is arranged inside the housing 204 and configured to perform an analysis on the urine collected through the collection port 218. Station 200 comprises a receiving compartment 212, located inside housing 204. Reception compartment 212 is configured to at least partially receive cartridge 202.

In an embodiment, the cartridge 202 can be rotatably mounted about the axis of rotation X, once in position in the annular compartment 212. The receiving compartment 212 is then an annular compartment. The receiving compartment 212 typically extends over 360° and forms a groove configured to at least partially receive the cartridge 202.

Alternatively, the cartridge 202 can be inserted translationally into the station 200. The receiving compartment 212 then forms a longitudinal channel for at least partially receiving the cartridge 202.

Other forms of receiving compartment 212 and ways of inserting the cartridge 202 can of course be envisaged.

The test assembly 230 may comprise a pump, an injector and an analysis device. The pump draws urine from the collection port 218, the injector injects the urine into at least one chamber of the cartridge and the analysis device analyzes the urine. In an embodiment, each chamber houses at least one reagent and the analysis device obtains certain property values (for example, physical/chemical properties, such as color) from the reagents after they have come into contact with the urine. In an embodiment, the analysis device is an optical analysis device configured to analyze the optical properties of the reagent. Alternatively or additionally, the analysis device performs an optical or spectroscopic analysis directly on the urine to determine certain properties. The injector and cartridge can move relative to each other so that the injector can open (e.g., pierce) the chamber, for example using a needle or needle-like device. The operation of the device 100 will be described in more detail below.

Dimensions relating to cartridge 202 are disclosed in documents WO′909, WO′933 and WO′80X. The maximum dimension of the device 100 transverse to the axis of rotation X is less than 15 cm, or even less than 10 cm. The maximum dimension of the device along the axis of rotation X is less than 5 cm.

### Cartridge

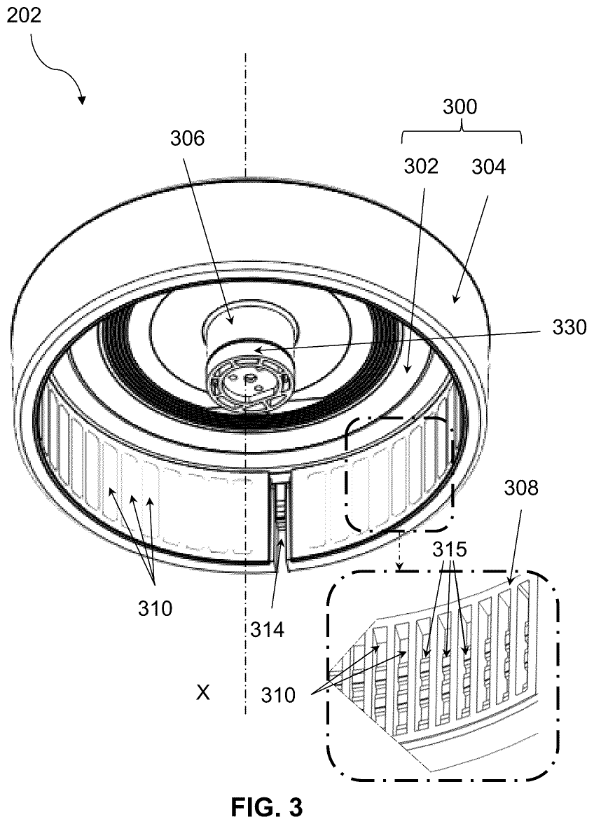

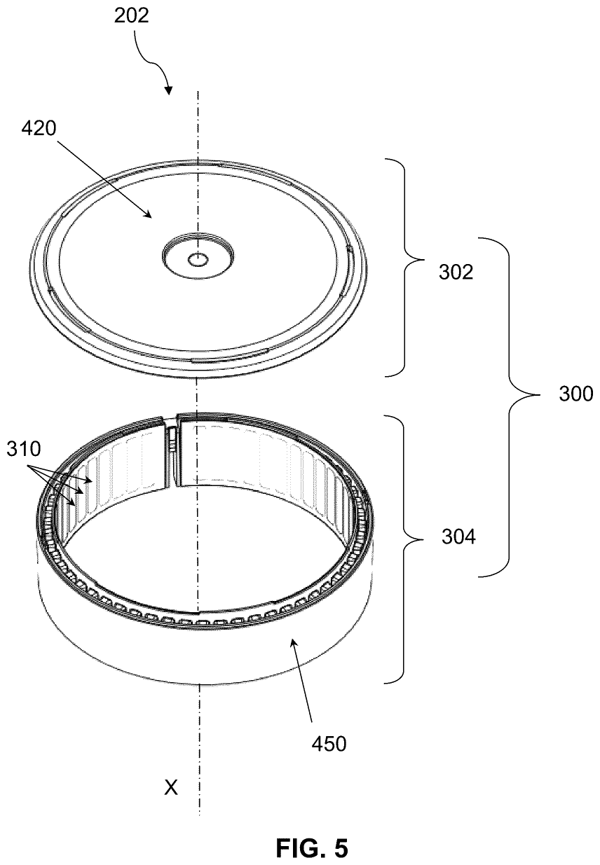

FIGS. 3 to 5, 7 and 8 show different embodiments of the cartridge 202. Cartridge 202 comprises a support 300 and a plurality of chambers 310.

The cartridge 202 may comprise at least ten chambers 310, in particular at least twenty-five chambers 310, or even at least fifty chambers 310.

Each chamber 310 is adapted to receive urine for urine analysis. In an embodiment, each chamber 310 houses a reagent 315 configured to react in a specific way on contact with the user's urine. Alternatively, urine injected into the chamber can be analyzed directly, for example by optical or spectroscopic analysis. Each chamber 310 is initially moisture-tight. By “moisture-tight chamber” it is meant a chamber that is impervious to moisture from outside the chamber. Thus, each chamber 310, and possibly the associated reagent 315, are fluidly separated from the rest of the cartridge 202. The reagent is arranged on a test strip, for example. Each chamber 310 then comprises one or more test strips. Alternatively, reagent 315 may be a liquid reagent received in the respective chamber.

A chamber 310 is notably formed by walls, which may be made of one or more materials. Document EP4338839 describes methods of making chambers 310. In particular, moisture-tight chambers 310 can be formed by superimposing layers of different materials.

Each moisture-tight chamber 310 comprises a cover 610 which can be pierced, for example by a needle, to enable urine to be injected by the injector of the test assembly 230. In an embodiment, the cover 610 is transparent or translucent to allow the passage of light inside the chamber and/or to perform optical analysis, and thus enable analysis of the result of the reaction of the reagent with the urine or direct optical analysis. Once pierced, chamber 310 loses its moisture-tightness and is then in fluid communication with the outside and therefore with the inside of the station.

The plurality of chambers 310 is held in position by the support 300. In other words, the support 300 mechanically holds the chambers 310 in position between them when the cartridge 202 is manipulated by a user during its insertion into or removal from the station 200, as well as during cooperation between the station 200 and the cartridge 202 when the device 100 is in operation. The support 300 is therefore the structural element that ensures the physical integrity of the cartridge 202. Support 300 supports chambers 310 and enables mechanical cooperation with station 200. During normal use of the cartridge 202 and the device 100, the chambers 310 are fixed to the support 300 and do not move relative to the support 300.

In particular, the support 300 is rigid. By “rigid”, it is understood that the support resists the stresses usually exerted by a user and by the station during normal use of the device. However, the support 300 may have a certain degree of flexibility, in particular a flexibility inherent in the materials, but which is not of functional use.

In an embodiment, a portion of the support 300, in particular a portion of the support 300 adjacent to a chamber 310, is transparent or translucent, to allow the passage of light inside the chamber 310 and/or to perform optical analysis in the chamber 310, and thus allow analysis of the result of the reaction of the reagent with the urine or direct optical analysis.

At least one portion of the support or support structure 300, called the desiccant portion 420, is composed of a solid desiccant. The solid desiccant is able to absorb moisture from the air in contact with the desiccant portion 420. In particular, the desiccant portion 420 has a water absorption capacity between 100 mg/g and 300 mg/g, especially between 140 mg/g and 200 mg/g. In particular, the solid desiccant comprises a hygroscopic material, i.e. a material which has the ability to absorb moisture from the air. In other words, the solid desiccant is configured to absorb water vapor molecules on contact with the desiccant portion 420.

In an embodiment, the solid desiccant is a mixture of at least two materials, in particular a mixture of a plastic polymer and a desiccant material. The desiccant material may, for example, be in the form of a powder diluted in the plastic polymer. The solid desiccant is made of zeolite-filled polypropylene, for example.

The desiccant portion 420 is located outside the moisture-tight chambers 310. In other words, there is no fluid communication between the internal volume of the moisture-tight chambers 310 and the desiccant portion 420. More precisely, there is no contact between the desiccant portion 420 and the internal volume of the chambers 310 when the chambers 310 are closed by the cover 610.

The desiccant portion 420 therefore has no desiccant function in the moisture-tight chambers 310 to preserve the integrity of the reagents prior to their use in the station 200. To this end, cartridge 202 may also include an additional desiccant disposed in at least one moisture-tight chamber 310. Beneficially, an additional desiccant is arranged in each moisture-tight chamber 310. The additional desiccant is therefore different from the desiccant portion 420. The additional desiccant is, for example, a strip of desiccant material. Alternatively or additionally, the additional desiccant is, for example, at least part of a wall of chamber 310 formed from a desiccant material.

The desiccant portion 420 may represent at least 25%, for example 50%, of the surface of support 300. In other words, at least 25%, for example 50%, of the outer surface or external boundary surface of support 300 is composed of the solid desiccant. In this way, the desiccant portion 420 represents a significant part of the support 300 and therefore offers high desiccant power without taking up additional space.

The support 300 thus has a dual function. The support 300 supports and holds in position the chambers 310 and therefore the reagents 315 in the cartridge 202, while helping to reduce moisture in the station 200. Indeed, as explained above, when the cartridge 202 is inserted into the station 200 and the chambers 310 are pierced, the urine injected into chamber 310 can subsequently evaporate and escape out of chamber 310. The desiccant portion 420 is then able to absorb at least some of this additional moisture. The desiccant portion 420 thus prevents repeated injections of urine into the chambers from increasing moisture in the urine analysis device too much, and thus preserves the proper operation of the electronic devices of the station 200.

As will be described later, the desiccant portion 420 can be a part of the support 300 (or may be integrally formed as part of the support 300) or a portion of a part of the support 300.

In an embodiment, the desiccant portion 420 forms a base on which the chambers 310 are mounted. In this way, the chambers 310 rest on the base. In other words, at least one face of the moisture-tight chambers 310 is in contact with the base, either directly or via a receiving portion. Thus, at least one exterior face of each moisture-tight chamber 310 is positioned in contact with, or supported by, the base, ensuring both mechanical stability of the chambers and exposure of the desiccant portion to ambient air.

Alternatively or additionally, the desiccant portion 420 comprises a force-receiving zone 306 from an actuator 330 of station 200. The force-receiving zone 306 is a zone of mechanical cooperation between the cartridge 202 and the station 200, for example to rotate and/or translate the cartridge 202 relative to the station 200 during operation of the device 100, in particular to move the chambers 310 past the injector and/or analysis device of the test assembly 230. This movement may be employed, for example, to index successive chambers 310 past an injector and/or an analysis unit of the test assembly 230. The desiccant portion 420 therefore both reduces moisture in station 200 and acts as a mechanical link between cartridge 202 and station 200. The force-receiving zone 306 can be a hub capable of receiving a shaft that can be rotated to actuate the cartridge.

In an embodiment, at least one portion of the support 300, referred to as the transparent portion 450, is made of a transparent or translucent material. The transparent portion 450 is made of a plastic material, for example. The transparent portion 450 can form at least part of the chambers 310 to allow light to pass through and reactions to be analyzed.

In an embodiment, the desiccant portion 420 and the transparent portion 450 form two separate parts of the support 300, assembled together. For example, the desiccant portion 420 and the transparent portion 450 are glued or clipped together. In an embodiment, the desiccant portion 420 and the transparent portion 450 form the entire support 300.

### Rotating Support

With reference to FIGS. 3 to 6, support 300 is, for example, a rotating support configured to be driven in rotation by station 200.

In an embodiment, the rotary support 300 has the shape of a straight circular cylinder, at least 80% of the shape of a hollow cylinder extending annularly around an axis which, when the cartridge 202 is mounted in the station 200, is the axis of rotation X.

As shown in FIG. 2, the receiving compartment 212 of the station 200 then has the shape of an annular compartment.

The rotatable support 300 may comprise an annular portion 302 and a cylindrical portion 304, which extends from a radially outer end of the annular portion 302. When the cartridge 202 is inserted into the station 200, the cylindrical portion 304 is housed inside the annular compartment 212.

In an embodiment, the annular portion 302 forms the desiccant portion 420. The desiccant portion 420 is substantially disk-shaped. This shape optimizes the ratio between the contact surface with the air and the volume occupied by the disk. For example, the disk has a diameter between 3 cm and 10 cm. The disc has a thickness between 1 mm and 10 mm. The desiccant portion 420 can form the base.

In this embodiment, the cylindrical portion 304 can form the transparent portion 450. Alternatively, the cylindrical portion 304 can be made of opaque material.

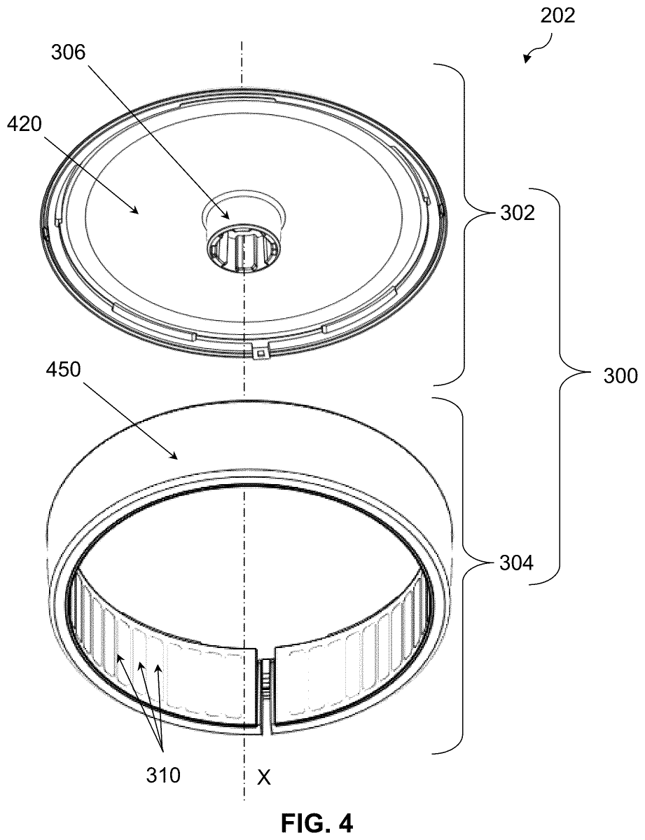

As shown in FIGS. 3 and 4, each chamber 310 can extend in a main direction orthogonal to the base. The chambers 310 are positioned along the cylindrical portion 304, so as to be able to pass selectively and/or successively in front of the injector and analysis device. The cylindrical portion 304 then acts as a receiving portion. The chambers 310 are arranged side by side in the shape of a right circular cylinder of at least 80% of the circle. The chambers 310 are all equidistant from the axis of rotation X, so that the injector can selectively inject urine once the desired moisture-tight chamber 310 is positioned at the desired location facing the injector. The injector can move towards the moisture-tight chamber 310 and pierce the cover 610 closing the moisture-tight chamber 310. A drain hole 314 is provided in the rotating support 300 to allow urine to drain from the injector to the outside of the device 100, via the drain hole 314 on the housing 204.

In the illustrated example, the annular portion 302 of the rotary support 300 remains outside the annular compartment 212 to reinforce the cylindrical part and/or rotate the cartridge 202. To this end, the annular portion 302 may comprise the force-receiving zone 306, for example in the form of a mechanical coupling (here a hub) which cooperates with the actuator 330 of the station (here a shaft). The force-receiving zone 306 is, for example, in the form of a shaft housed in a cavity 250 of the station 200.

### Support Variants

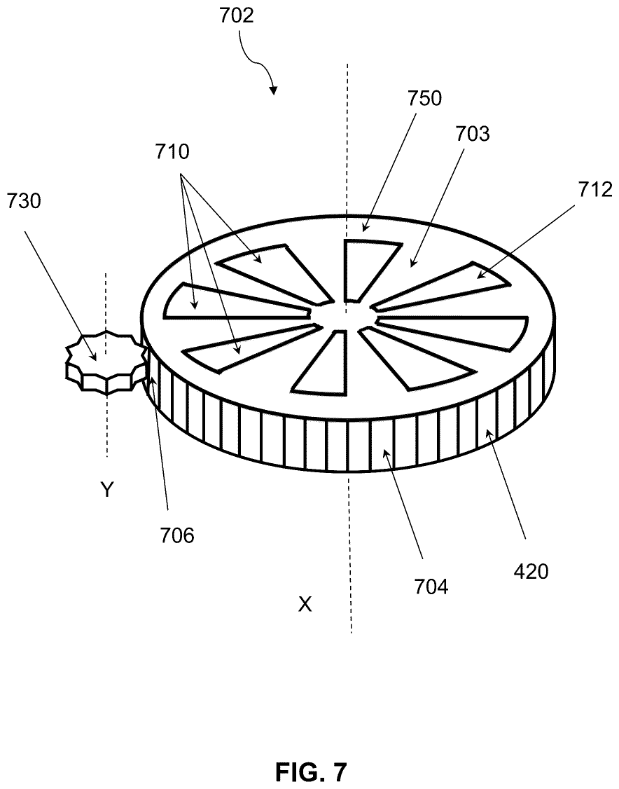

Alternatively, with reference to FIG. 7, the cartridge 702 has a solid cylindrical shape. The receiving compartment 212 of station 200 then has a shape complementary to the cylinder. The rotary support 300 may comprise an annular part 703 and a cylindrical part 704, which extends radially inside the annular part 703. Each moisture-tight chamber 710 may extend orthogonally to the direction of rotation X. Each chamber 710 can thus extend radially, between the cylindrical part 704 and the center of the annular part 703. The desiccant portion 420 is formed by the cylindrical part 704, as described above. The annular part 703 can form the transparent portion 750. Each chamber 710 is closed by a cover 712 placed over the annular part 703. In operation, the analysis device and injector are positioned above and/or below the cartridge 202 along the axis of rotation X. The desiccant portion 420 here comprises a force-receiving zone 706 in the form of teeth operable by an actuator 730 in the form of a gearwheel. Rotation of the actuator along a Y axis parallel to the X axis causes the cartridge 702 to rotate, so that the chambers 710 pass in front of the injector and/or analysis device.

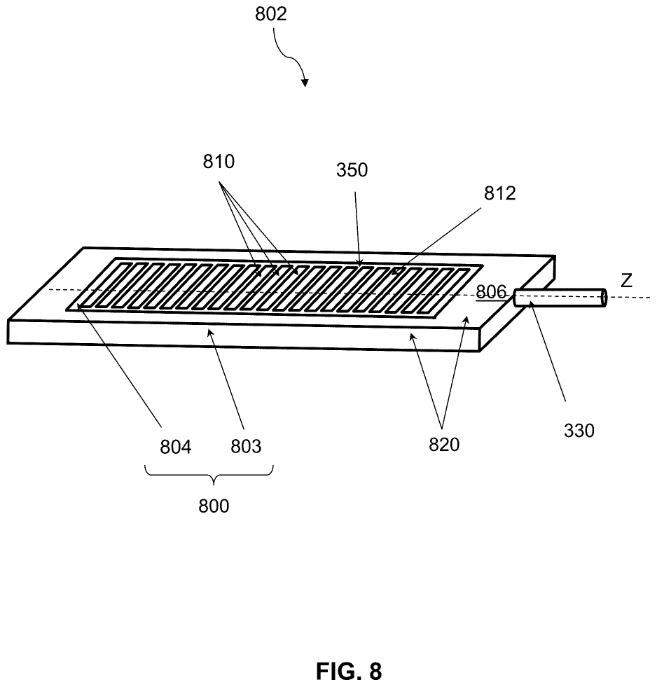

Alternatively, with reference to FIG. 8, the cartridge 802 has a parallelepiped shape extending mainly along a main Z direction. The receiving compartment 212 of the station 200 then has a channel shape, complementary to the cartridge 802. The support 800 may comprise a base 803 and, in an embodiment, a receiving portion 804, suitable for receiving the moisture-tight chambers 810. Each chamber 810 can extend orthogonally to the main Z direction. The chambers 810 extend parallel to each other. The desiccant portion 820 is formed by the base 803. Each chamber 810 is closed by a cover 812. The cover 812 and/or the receiving portion 804 may be transparent. In operation, the analysis device and injector are arranged above and/or below the chambers 810, in a direction orthogonal to the main Z direction. The desiccant portion 820 here comprises a force-receiving zone 806 in the form of a wall suitable for being pushed or pulled by an actuator 330 in the form of a rod extending along the main direction Z and driven, for example, by a stepper motor. Translating the actuator 330 in the main Z direction causes the cartridge 802 to translate, so that the chambers 810 pass in front of the injector and/or analysis device.

### Station-Cartridge Interaction

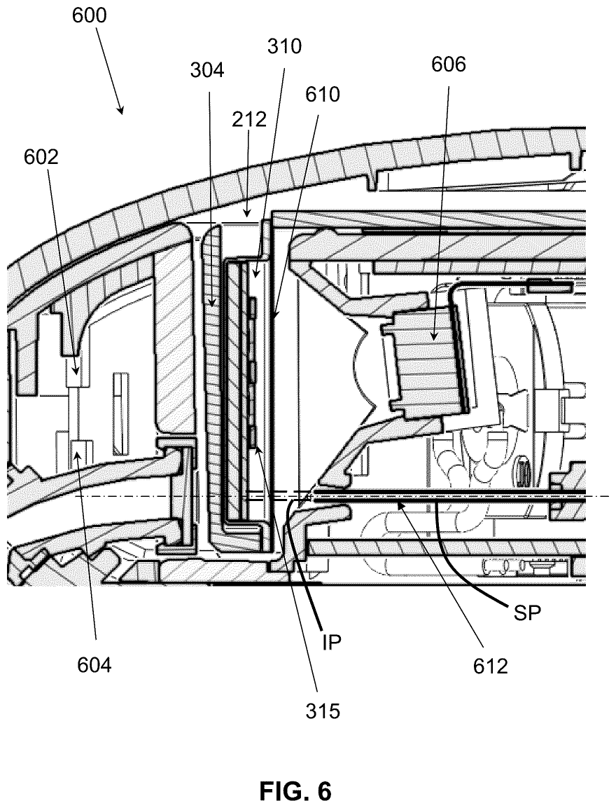

FIG. 6 shows in greater detail the interaction between the cartridge 202 as shown in FIGS. 3 to 5 and the station 200 when or after the injector has been activated. The analysis device 600 comprises at least one light source 602, 604 (for example, two light sources; in particular, four light sources) and at least one optical sensor 606. Light travels from the light source 602, 604 to the optical sensor 606, passing through the cartridge 202 and in particular the cylindrical portion 304 and possibly the reagent 315.

In an embodiment, analysis device 600 is configured to measure the absorbance of a portion of reagent 315.

The absorbance is detected by the light source (e.g. an LED), which can pass light through the strip, and the optical sensor, which receives the spectrum with around ten wavelengths.

In an embodiment, the light sensor is a camera capable of detecting a change in color, in particular a change in color intensity, of some of the reagents 315.

The camera can detect color in RGB values, for example.

The injector comprises an injection end 612 (e.g. a needle), which can be moved between a standby position SP and an injection position IP. In the standby position SP, the injection end 612 is outside the cartridge 202 (in a radially innermost position), so that the cartridge 202 can rotate freely in the annular receiving compartment 212. In an injection position IP, injection tip 612 has pierced cover 610 to access the interior of chamber 310 and can inject a little urine onto reagent 315.

In the SP position, the injector is located radially inside the annular chamber. This maximizes the radius of the annular chamber while minimizing the size of station 200.

In an embodiment of the test assembly 230 disclosed above, the skilled person will understand that each test set configured to analyze the urine sample collected through the collection port 218 can be arranged inside the housing 204.

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

The articles “a” and “an” may be employed in connection with various elements and components, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

1. A urine analysis cartridge comprising:
a support,
a plurality of moisture-tight chambers, each moisture-tight chamber being suitable for receiving urine for urine analysis,
wherein:
the plurality of moisture-tight chambers is held in position by the support,
at least a portion of the support, forming a desiccant portion, is composed of a solid desiccant, capable of absorbing moisture from the air in contact with the desiccant portion, the desiccant portion being outside the moisture-tight chambers.

2. The cartridge according to claim 1, wherein the solid desiccant is a hygroscopic material.

3. The cartridge according to claim 1, wherein the solid desiccant is a bi-material mixture of a plastic polymer and a desiccant material.

4. The cartridge according to claim 1, wherein the solid desiccant is formed from zeolite-filled polypropylene.

5. The cartridge according to claim 1, wherein the desiccant portion has an absorption capacity between 100 mg/g and 300 mg/g.

6. The cartridge according to claim 5, wherein the desiccant portion has an absorption capacity between 140 mg/g and 200 mg/g.

7. The cartridge according to claim 1, wherein the desiccant portion represents at least 25% of a surface of the support.

8. The cartridge according to claim 1, wherein the desiccant portion represents at least 50% of a surface of the support.

9. The cartridge according to claim 1, wherein the desiccant portion is rigid.

10. The cartridge according to claim 1, wherein the desiccant portion comprises a force-receiving zone of an actuator of a station.

11. The cartridge according to claim 1, wherein the desiccant portion forms a base on which the chambers are mounted.

12. The cartridge according to claim 1, wherein each chamber comprises a pierceable cover which can be pierced.

13. The cartridge according to claim 12, wherein each cover can be pierced by a needle.

14. The cartridge according to claim 1, wherein the support is a rotating support, the chambers being arranged side by side in the shape of a right circular cylinder of at least 80% of a circle.

15. The cartridge according to claim 14, wherein the desiccant portion substantially forms a disk.

16. The cartridge according to claim 1, wherein the cartridge further comprises an additional desiccant, different from the desiccant portion, disposed in at least one moisture-tight chamber.

17. The cartridge according to claim 16, wherein the additional desiccant is a strip of desiccant material.

18. The cartridge of claim 1, wherein the desiccant portion defines a force-receiving zone comprising a hub configured to couple with a drive member of an analysis station to transmit rotational and/or translational motion that indexes the chambers past an injector and/or an analysis unit, the hub being formed of the solid desiccant material such that the force-receiving zone simultaneously provides mechanical coupling and ambient-humidity sorption.

19. A urine analysis device comprising:
a cartridge according to claim 1,
a station configured to be positioned on a wall of a toilet bowl, the station comprising:
a housing comprising a receiving compartment in which the cartridge is at least partially received,
an injector, configured to inject urine into one of the moisture-tight chambers,
an analysis device for analyzing the urine injected into the moisture-tight chamber.

20. The device according to claim 19, wherein the cartridge is removably arranged in the housing.
